(19) United States
(12) Patent Application Publication
(10) Pub. No.: US 2025/0308696 A1
(43) Pub. Date: Oct. 2, 2025

(54) AN ARTIFICIAL INTELLIGENCE ENABLED WEARABLE ECG SKIN PATCH TO DETECT SUDDEN CARDIAC ARREST

(71) Applicant: TOPIA LIFE SCIENCES LIMITED, London (GB)

(72) Inventor: Kamleshkumar PATEL, London (GB)

(21) Appl. No.: 19/109,347

(22) PCT Filed: Sep. 7, 2023
(86) PCT No.: PCT/GB2023/052308

§ 371 Date: Mar. 6, 2025

(30) Foreign Application Priority Data
Sep. 7, 2022 (GB) 2213074.4

(51) Int. Cl. G16H 50/20; A61B 5/00; A61B 5/024; A61B 5/0245; A61B 5/257; A61B 5/265; A61B 5/308; A61B 5/352; A61B 5/366; G16H 10/60; G16H 40/67

(52) U.S. Cl. CPC G16H 50/20; A61B 5/0006; A61B 5/02405; A61B 5/02438; A61B 5/0245; A61B 5/257; A61B 5/265; A61B 5/308; A61B 5/352; A61B 5/366; A61B 5/6833; G16H 10/60; G16H 40/67; A61B 2562/164; A61B 2562/166

(57) ABSTRACT

There is described an artificial intelligence wearable ECG skin patch (400) to detect sudden cardiac arrest. The wearable ECG monitoring patch (400) with AI based predictive analytics and remote based cardiac monitoring (615) system that can detect cardiac arrhythmias automatically in real-time and make a diagnosis with AI models trained with acquired data. The wearable skin has a biocompatible polymer patch (400) which captures the electrical signal through a flexible printed electronic technology based conducting ink and a substrate. The microcontroller controls (201), store and transmit the data packets. The IoT connected signal transmission is capable of recording and transferring the data packets through wireless communication. The AI engine is capable of analysing, evaluating, testing and providing the data packets of sudden cardiac arrest through a peak detector algorithm. The ECG skin patch (400) to detect and measure the sudden cardiac arrest with the R-R interval time series to obtain heart rate variability.

### FIELD OF THE INVENTION

The present invention relates to an artificial intelligence wearable ECG skin patch to detect sudden cardiac arrest. More particularly it relates to wearable electrocardiogram (ECG) monitoring patches with artificial intelligence (AI) based predictive analytics and remote based cardiac monitoring system that is capable to detect cardiac arrhythmias automatically in real-time and make a diagnosis with AI models trained with acquired data.

### BACKGROUND OF THE INVENTION

A cardiac monitoring generally refers to continuous or intermittent monitoring of heart activity, generally by electrocardiography, with an assessment of the patient's condition relative to their cardiac rhythm and it is different from hemodynamic monitoring, which monitors the pressure and flow of blood within the cardiovascular system. The two may be performed simultaneously on critical heart patients. The cardiac monitoring with a small device worn by an ambulatory patient is known as ambulatory electrocardiography. The transmitting data from a monitor to a distant monitoring station is known as telemetry or biotelemetry.

The leading cause of heart disorder is Arrhythmia which is categorized into three types namely premature heartbeat, tachycardia, and bradycardia where most of the arrhythmias does not present any risk immediately and happens usually in our daily life. Acute stroke is mainly caused by atrial fibrillation and sudden shock or cardiac death is caused mainly due to ventricular tachycardia.

According to statistics taken worldwide by the World Health Organization, cardiovascular diseases (CVDs) are the leading cause of death globally. An estimated 17.9 million people died from CVDs in 2019, representing 32% of all global deaths. Of these deaths, 85% were due to heart attack and stroke. Over three quarters of CVD deaths take place in low- and middle-income countries. Out of the 17 million premature deaths (under the age of 70) due to no communicable diseases in 2019, 38% were caused by CVDs. Most cardiovascular diseases can be prevented by addressing behavioural risk factors such as tobacco use, unhealthy diet and obesity, physical inactivity and harmful use of alcohol. It is important to detect cardiovascular disease as early as possible so that management with counselling and medicines can begin.

Among cardiovascular diseases, Arrhythmia is the most important cause for death hence aged community people can be given continuous health care by utilizing wearable devices for monitoring and detection of unusual electrocardiogram (ECG) signals such that instant warning messages can be sent to hospital or concerned medical practitioner. Based on alert messages immediate care is given to the patient avoiding tragedies happening. In present invention several arrhythmias are focused for building an algorithm based on convolutional neural network (CNN) for the classification of cardiac disease. This health care platform of Artificial Intelligence (AI) involves IoT based wearable hardware, cloud database and user interface application.

The development of a smart wearable ECG monitoring patch with AI based predictive analytics and remote monitoring. The present invention works on conceptualization and development of the product prototype based on the principles of ECG, Internet of Things (IoT) and Artificial Intelligence.

US20190313968A1 discloses a wearable, individual, customized and different sized technology for men and women, composed of electrodes, conductive track and airtight container for medicines, coupled to one another, and a mini electrocardiogram (ECG) apparatus containing a GSM (Global System Mobile) modem and a GPS (Global Positioning System) or a Bluetooth system which, through a wireless network specification within a personal scope (Wireless Personal Area Networks—PANs) deemed as PAN-type or even WPAN, the electrical signal acquisition begins when the user press the button down. It is in the field of medical, recreational and/or sport applications, aiming at monitoring patients at high cardiovascular risk, being possible to diagnose it as soon as possible, aiming at shortening time to definite treatment of those who present acute coronary syndrome (ACS), acute myocardial infarction (AMI), acute atrial fibrillation-type (AAF) cardiac arrhythmias, and other cardiac arrhythmias or other cardiac pathologies capable to be detected by the electrocardiographic trace.

WO2019073288A1 discloses a remote ECG monitoring and alerting, based on a wearable ECG patch, intermediate device, cloud server and a monitoring device. Configuration of the sensing device allows transmission of ECG samples via personal area network to the nearby intermediate dew server. The intermediate device is realized by a small mobile device (smartphone, smart watch, tablet or other device), with an application that accepts the sensed signals, displays the ECG data in a user-friendly manner, transmits captured data to a cloud server and alerts in case of a detected abnormal heart function. The cloud server is used for further processing and data sharing and a monitoring device is used to visualize the ECG data. The caregiver and/or doctor can analyse the ECG data for reference and personalized medical advice, based on relevant data, can be provided.

KR102309022B1 discloses an artificial intelligence based remote monitoring system for bio-signals, which enables real-time monitoring of received multiple bio-signals and requests to be read with deep learning artificial intelligence through a heart disease analysis server and prognostic analysis server to detect heart disease If prognostic analysis is performed and the pre-set alarm threshold is exceeded, an alarm message is sent to the hospital's monitor system, medical staff, and patient's mobile terminal, and the type of bio signal or measurement cycle of multiple bio signal measuring devices is determined according to the patient's severity or prognosis. It can be set manually or automatically remotely.

The applications of wearable devices for disease monitoring and prediction of adverse events with alerts to inform the patient/caregiver is growing day by day. The recent developments in the wearable technology domain allows the doctors, scientists, engineers and entrepreneurs to work together in providing new and innovative solutions for effective monitoring and diagnosis of various ailments.

More than 50% of overall deaths occurring due to cardiovascular related diseases is due to sudden cardiac arrest (SCA). The existing clinical prediction models (CPM) fail miserably in predicting the event of sudden cardiac arrest (SCA) occurrence among the vulnerable population of pre-existing cardiac abnormalities or even normal. Most sudden cardiac arrest (SCA) related deaths occur due to misdiagnosis and unavailability of emergency hospital care. There is a substantial lack of information and insufficient understanding as related to accurate prediction of sudden cardiac arrest (SCA) and prevention of sudden cardiac deaths.

An information retrieval from long term and complete Electrocardiography (ECG) profiles in association with circadian rhythm can be helpful in detecting the probability of occurrence of sudden cardiac arrest. The proper diagnosis of the symptoms along with an artificial intelligence (AI) based expert system can address the gap in prediction of SCA and assist the patient or doctor in execution of his/her proper treatment plan.

In conventional technology, the ECG is detected through large and stationary equipment in professional medical institutions. The kind of equipment usually employs ten electrodes to collect twelve lead ECG data due to their good performance in short-term measuring. However, the equipment is unlikely to be portable, which means that patients' activities are severely limited during the period of data collection. Moreover, as these devices are usually too expensive for home use, patients have to go to the hospital frequently, which inevitably increases the burden of hospitals. Therefore, a portable system for long-term ECG signal detection with low costs is highly desired.

Also, in the current scenario no device has yet hit the market that has the potential to disrupt the traditional ECG acquisition and cardiovascular diagnosis market. There is a significant development in intellectual resources and demonstration of wireless ECG patches in academia. Most of the existing devices are suitable for remote monitoring but don't come with AI enabled disease diagnosis.

To solve the above-mentioned problems related to the conventional technology, the inventor of the present disclosure came up with a new innovation which is wearable ECG devices with IoT and AI for smart healthcare. Most of the portable ECG devices in the market come with short time monitoring to calculate R-R interval based heart rate from the device module. The data coming out of these pocket ECG devices are not used for training AI models to provide smart diagnostic services. Most of them are suitable for only remote monitoring and manual diagnosis. To address these issues, the present disclosure considers integrating the hardware with the software in a cloud platform so as to complete monitoring and diagnostics solutions as well. This can be a breakthrough device that can save millions of lives lost due to improper or late diagnosis of cardiac arrest. Sudden heart attacks seriously threaten the lives of cardiac patients, especially when patients are alone. Therefore, disease warning on the IoT cloud has become important for protecting patients from being injured. Based on the results of data analysis, the IoT cloud is able to understand the real-time health conditions of the patient. With the aid of these systems, long-term ECG can be monitored in a cost-effective manner within house or hospital environments.

### OBJECT OF THE INVENTION

The principle object of the present invention is to overcome all the above mentioned and existing drawbacks of the prior arts by providing an artificial intelligence enabled wearable ECG skin patch to detect sudden cardiac arrest.

Another object of the present invention is to provide an artificial intelligence enabled wearable ECG skin patch to detect sudden cardiac arrest being capable to automatically detect cardiac arrhythmias in real-time and make diagnosis with AI models trained with acquired data.

Another object of the present invention is to provide an artificial intelligence enabled wearable ECG skin patch to detect sudden cardiac arrest being capable to calculate R-R interval based heart rate through the AI module.

Another object of the present invention is to provide an artificial intelligence enabled wearable ECG skin patch to detect sudden cardiac arrest which is a quick, accurate and reliable prediction of abnormal heart rhythms and trigger alert response for the patient/caregiver from the exact point of care.

Yet another object of the present invention is to provide an artificial intelligence enabled wearable ECG skin patch to detect sudden cardiac arrest being capable for automated prediction of any sudden cardiac events detrimental to the smooth functioning of the cardiovascular system.

Another object of the present invention is to provide an artificial intelligence enabled wearable ECG skin patch to detect sudden cardiac arrest being capable of capturing the electrical signals on the skin emanating from the heart using a flexible printed electronics technology based conducting ink and substrate.

Another object of the present invention is to develop a smart wearable skin patch with integrated circuits to transmit, store, process and analyse electrical signals of the heart for computer aided diagnostic applications.

Another object of the present invention is to develop a system, using Artificial Intelligence (AI) and Internet of Things (IoT) technology, for effective monitoring and diagnosis of underlying cardiac abnormality.

Another object of the present invention is to provide a flexible dry electrodes which can be printed on Thermoplastic Polyurethane (TPU) substrate and laminated with breathable textile material.

Yet, another object of the present invention is to provide an analog front end (AFE) circuit capable of capturing low amplitude multi-resolution signals which are captured by the said wearable patch.

Another object of the present invention is to provide an AFE being responsible for acquiring the analog signal using inbuilt instrumentation amplifiers designed to acquire bio-potentials.

Yet another object of the present invention is to provide a wireless connection.

Another object of the present invention is to provide a microcontroller unit capable of controlling, storing and transferring the data of Patient. The microcontroller is responsible for controlling the AFE, ADC and wireless signal transmission via Bluetooth and Wi-Fi.

Another object of the present invention is to provide a crystal oscillator which being capable to provide the clock frequency for data flow synchronisation.

Yet another object of the present invention is to provide a rechargeable battery being capable to powering up to the PCB. The battery charger circuit being capable to provide a battery charger circuit with battery level indicator.

Yet another object of the present invention is to provide a LED which being capable to provide indication and track the sudden cardiac attack.

Another object of the present invention is to provide a voltage regulator capable of providing a supply of necessary voltage to the ICS.

Yet another object of the present invention is to provide a USB to UART converter IC that is used to flash the code into the microcontroller.

Another object of the present invention is to provide a jack being connected to an electrode by snap connector.

Another object of the present invention is to provide an artificial intelligence enabled wearable ECG skin patch to detect sudden cardiac arrest having a dry cell arrangement being capable to recharge the battery to reduce the overhead on the patient.

Yet another object of the present invention is to provide an artificial intelligence enabled wearable ECG skin patch to detect sudden cardiac being capable for data storing, processing and analytics a cloud computing infrastructure is realised with virtual servers and databases with Hypertext Transfer Protocol Secure (https) and Message Queuing Telemetry Transport (mqtt) based communication protocols.

Yet another object of the present invention is to provide a mobile application which can be compatible with all major mobile operating systems to view the acquired signal in real time using Bluetooth communication. The WI-FI mode being capable to connect said device to transmit and stored the data into cloud servers and the SD card mode being capable to store data into the PC/Laptop through USB connection.

One more object of the present invention is to provide an artificial intelligence model being capable for probabilistic estimation of cardiac health and a trigger to alert the patient in case of arrhythmia and chance of a sudden cardiac arrest.

Another object of the present invention is to provide a 1D discrete Wavelet Transforms (DWT) being capable for signal de-noising.

Yet another object of the present invention is to provide an AI Machine Learning (ML) pipeline that being capable for data pre-processing, feature extraction, selection, training AI & ML models, validation and testing, performance evaluation and deployment in the web server.

Another object of the present invention is to provide a deep learning which is providing direct training through the Convolutional Neural Network (CNN).

Another object of the present invention is to provide an ECG patch being capture the entire span of the heart strictly in accordance with the principles of Einthoven Triangle.

Yet another object of the present invention is to provide a peak detector algorithm which is capture the instantaneous heart rate from the R-peaks of the ECG and also plot the R-R interval time series to obtain Heart Rate Variability (HRV). Also, detecting arrhythmia conditions and alert the patient under abnormal conditions.

Another object of the present invention is to provide a non-linear discrete dynamical systems theory (ND-DST) in mathematical modelling to quantify the underlying cardiovascular dynamics for effective monitoring of the condition of the heart.

Another object of the present invention is to provide an AI model being capable to detect the Tachycardia (HR >100 BPM), Atrial Fibrillation (Afib—Chaotic), Atrial Flutter (AFL—Impulsive), Bradycardia (HR <60 BPM) and Ventricular Fibrillation (VF).

Another object of the present invention is to provide an artificial intelligence enabled wearable ECG skin patch to detect sudden cardiac being capable to replace the existing ECG machine, Holter recording machine and ECG stress test protocols with an AI enabled smart wearable device as proposed here.

Yet another object of the present invention is to provide a pins through the device being capable to reusable with provision for further firmware updates for up-gradation via offline and online modes.

Another object of the present invention is to provide an ECG skin patch is an integrated expert system that can work in assisting the patient's physician or cardiologist for secondary level of diagnosis and treatment planning.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

A brief summary describing the various embodiments incorporated, and/or may be incorporated, in the overall system design of an AI enabled expert system comprising smart skin patches for automated prediction of any sudden cardiac events detrimental to the smooth functioning of the cardiovascular system. The overall system can be classified into three major embodiments, namely wearable skin patches, IoT connected signal transmission unit and AI engine

The present disclosure relates to an artificial intelligence enabled wearable ECG skin patch to detect sudden cardiac arrest.

An aspect of the present disclosure is to provide an artificial intelligence enabled wearable ECG skin patch (400) to detect sudden cardiac arrest, the skin patch (400) comprising an IoT connected signal transmission unit and an artificial intelligence engine; wherein, said wearable ECG skin patch (400) comprises a flexible printed electronic technology based biocompatible polymer ECG skin patch (400) that is capable of capturing an electrical signal; said IoT connected signal transmission unit comprising a microcontroller unit (201) that is capable of controlling signal transmission using a wireless interface; said artificial intelligence engine comprising an artificial intelligence (AI) and Machine Learning (ML) pipeline that is arranged to perform a sequence of steps comprising: a data pre-processing step, a feature extraction step, a feature selection step, a training step, a validation and testing step, and a performance evaluation step; characterized in that, said wearable ECG skin patch (400) is capable of capturing the entire span of the heart to detect the sudden cardiac arrest; and in that a sudden cardiac arrest is predicted through said AI and ML pipeline; wherein the AI and ML pipeline is trained using a knowledge database and is thereafter arranged to automate the process of cardiac disease prediction; wherein a peak detector algorithm of the artificial intelligence engine is capable of capturing the instantaneous heart rate from the R-peaks of the ECG so as to obtain a measure of Heart Rate Variability (HRV). In this system an automated AI pipeline is proposed which is capable of signal cleaning, segmentation and event detection. On detection of abnormal cardiac events, the system would trigger the response to the user in near real time and generate a complete ECG profile report mentioning the probabilistic chance of occurrence of a particular disease.

An aspect of the present disclosure is to provide an artificial intelligence enabled wearable ECG skin patch to detect sudden cardiac arrest, the skin patch comprising an IoT connected signal transmission unit, and an artificial intelligence engine; wherein, said wearable ECG skin patch is capable of (and/or arranged to) capture electrical signals through a flexible printed electronic technology based biocompatible polymer ECG skin patch. The IoT connected signal transmission unit comprises a microcontroller unit capable of (and/or arranged to) control the signal transmission through the wireless connectivity and said artificial intelligence engine comprises an artificial intelligence (AI) and Machine Learning (ML) pipeline that performs a sequence of steps including: a data pre-processing step, a feature extraction step, a feature selection step, a training AI and ML models step, a validation and testing step, and a performance evaluation step; characterized in that said wearable ECG skin patch is capable of (and/or arranged to) to capture the entire span of the heart to detect the sudden cardiac arrest; said sudden cardiac arrest being predicted through said AI pipeline, said AI pipeline being arranged to automate the process of accurate cardiac disease prediction from a knowledge database; a peak detector algorithm of said AI engine being capable of (and/or arranged to) capture an instantaneous heart rate from the R-peaks of the ECG and to (e.g. plot the R-R interval time series to) obtain a Heart Rate Variability (HRV).

Another aspect of the present disclosure is to capture the electrical signals on the skin emanating from the heart using a flexible printed electronics technology based biocompatible polymer patch. The dry electrodes are made of conducting Ag/AgCl ink printed on a Thermoplastic Polyurethane (TPU) substrate and laminated with breathable textile material.

Another aspect of the present disclosure is to provide an Analog Front End (AFE) designed to capture low amplitude multi resolution signals as captured by the wearable patch. The AFE is responsible for acquiring the analog signal using inbuilt instrumentation amplifiers designed to acquire biopotentials. Four electrode points are used to capture all the three primary leads of a 3-channel ECG signal monitoring scheme based on the Einthoven Triangle. In one of the embodiments of the integrated circuit this analog signal is sampled with its inbuilt 24 bit ΣΔ-ADC. The other embodiment is a microcontroller unit (MCU) with integrated Bluetooth low energy (BLE) module and Wi-Fi 2.5 GHz. This MCU is the heart of the IoT module and is responsible for controlling the AFE, ADC and wireless signal transmission via Bluetooth and Wi-Fi. In a certain embodiment, and present disclosure have incorporated a provision to embed a SD card module which stores the 3 channels of raw ECG data into the device for downloading later on.

Another aspect of the present disclosure is to provide a rechargeable Lithium ion battery (LiPo) to power up the device along with indicator Light Emitting Diodes (LEDs). The battery charger circuit with battery level indicator LEDs is installed with a USB interface.

Another aspect of the present disclosure is to provide two separate linear voltage regulators with diodes incorporated to provide necessary supply voltages to the respective ICS. In one embodiment, the USB to UART converter IC is used to flash the code into the microcontroller.

Another aspect of the present disclosure is to provide a two 3.5 mm jacks are used for connecting the electrodes using snap connectors. In one such aspect, the present disclosure has an alternative to replace the 3.5 mm jacks with shielded snap connectors with banana clip, hence reducing the size of the device.

Another aspect of the present disclosure is to power up the device using dry cell arrangement and hence reduce the overhead on the patient side of recharging the battery. Another aspect of the present disclosure is to provide a casing, with four snap connectors, to attach the PCB and battery unit along with the wearable skin patch. The casing contains the top cover with three switches for different modes of operation and indicator LEDs to show the device running status. In case of abnormal heart function the LED can be driven in a particular blinking frequency to alert the patient or caregiver.

Another aspect of the present disclosure is to provide data storing, processing and analytics a cloud computing infrastructure is realised with virtual servers and databases with Hypertext Transfer Protocol Secure (https) and Message Queuing Telemetry Transport (mqtt) based communication protocols.

Another aspect of the present disclosure is to provide a mobile application (Mobile App) designed compatible with all major mobile operating systems to view the acquired signal in real time using Bluetooth communication. The viewed signal can be recorded in the user's mobile phone inbuilt memory.

Another aspect of the present disclosure is to provide a recorded signal can be uploaded to the cloud database for storing, processing and AI analytics and there is provision to display the outcome of the AI model for probabilistic estimation of cardiac health and a trigger to alert the patient in case of arrhythmia and a sudden cardiac arrest.

Another aspect of the present disclosure is to provide an artificial intelligence enabled wearable ECG skin patch to detect sudden cardiac arrest which can be work without smartphones using a common Wi-Fi gateway to have direct access to the database using the same cloud server. In case of hospital monitoring this system can be beneficial for the patient as well as doctors to plan their treatment accordingly. In another aspect of the present disclosure, the web application (Web App) along with a dashboard to monitor in real time. The data here is stored in a cloud database and the backend AI pipeline starts working as soon as it gets the input to provide the probabilistic output about the severity of cardiac ailment. The dashboard will also have the provision to store the results for visualising later on.

Another aspect of the present disclosure is to enable the user to download the data from the integrated SD card embedded in the device itself using a USB cable and desktop application for offline data analysis. This will cover for the situations when a patient is advised to wear it for 7 days or more and during situations when he/she may not always be up with a smartphone or near to any secured Wi-Fi gateway to connect with the cloud. In such a scenario, this embodiment can help the user to acquire data continuously into the device and later on download the data in a laptop/personal computer.

In another aspect of the present disclosure, the signal de-noising using 1D discrete Wavelet Transforms (DWT) is modelled by manual observation of ECG signals. Once all the parameters have been chosen, the same DWT model is used during data pre-processing stages for de-noising of any baseline wanders or high frequency noise by removing the unnecessary coefficients without losing any of the relevant information. By adopting this embodiment, we eliminate the use of any frequency domain filters, hence no information loss due to domain transfer.

Another aspect of the present disclosure is to provide an AI and Machine Learning (ML) pipeline that contains a sequence of steps running in tandem namely, data pre-processing, feature extraction, feature selection, training AI and ML models, validation and testing, performance evaluation and deployment in the web server.

Another aspect of the present disclosure is to provide a deep learning model, such as a 1-D Convolutional Neural Network (CNN), that may be incorporated for direct training of the AI models using pre-processed data stored in cloud database (DB). The peak detector algorithm is employed to capture the instantaneous heart rate from the R-peaks of the ECG and also plot the R-R interval time series to obtain Heart Rate Variability (HRV). This embodiment improves the detection of arrhythmia conditions so as to alert the patient under abnormal conditions.

Another aspect of the present disclosure is to provide a nonlinear discrete dynamical systems theory (ND-DST) in mathematical modelling to quantify the underlying cardiovascular dynamics for effective monitoring of the condition of the heart.

Another aspect of the present disclosure is to provide a calculation of sample Entropy (SampEN), which is an Information Theory based parameter to capture the irregularities and complexities in ECG time series which is an effective indicator of rate of information transfer pertaining to a certain stimulus The fractal dimension as an indicator of the complexity and irregularity of heart beats from the complete ECG profile. Fractal Dimension (FD) or Higuchi's Fractal Dimension is very suitable for identifying intermittent clusters of PQRST arising during sudden cardiac arrest or tachycardia and bradycardia situations. The variation in complexity of the signal is a good indicator of underlying mechanisms of the heart as obtained from the electrical signals emanating on the skin surface. In certain embodiments, these parameters will be used along with the parameters as obtained from the traditional Pan Tomkin's algorithm to create a feature set for supervised training of ML models.

Another aspect of the present disclosure is to provide a feature set to be expanded with feature vectors obtained from multi-scale analysis of ECG signal channels namely, The multi-scale permutation Entropy (MPE) and Multi fractal Detrended Fluctuation Analysis (MFDA). The circadian rhythm based analysis has been incorporated where the heart rate parameters will be plotted along hourly intervals throughout one circadian cycle or more. The circadian rhythm based analysis has often been reliable in clinical prediction models of sudden cardiac arrest.

Another aspect of the present disclosure is to provide an output layer that is varied according to the requirements of the patient. The binary classification is employed to separate the normal from the abnormal. In some embodiments of the same ML model, a two-step process may be used where the model will give disease classification output in the second stage after it has identified the input as normal and abnormal in the first stage. This manipulation increases the efficiency of the AI model significantly and helps us to manifest certain embodiments that require immediate detection of sudden changes in the cardiac rhythms and automatically classify based on the training dataset.

Another aspect of the present disclosure is to provide an output layer of the AI model can classified to detect Tachycardia (HR >100 BPM), Atrial Fibrillation (Afib—Chaotic), Atrial Flutter (AFL—Impulsive), Bradycardia (HR <60 BPM) and Ventricular Fibrillation (VF).

Another aspect of the present disclosure is to provide a pin through the device capable of being (and/or arranged to be) reusable with provision for firmware updates for up-gradation via offline and online modes. The ECG skin patch is an integrated expert system that can work in assisting the patient's physician or cardiologist for secondary level of diagnosis and treatment planning and ECG skin patch is being capable to automatically plot the data in sync with the circadian rhythm.

Another aspect of the present disclosure is to provide the combinations of the other embodiments that would make it possible to replace the existing ECG machine, Holter recording machine and ECG stress test protocols with an AI enabled smart wearable device as proposed here.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings.

FIG. 1A shows the detailed schematic of wearable skin patch for 3 lead operations according to one embodiment.

FIG. 1B shows the wearable skin patch for single lead operation according to one embodiment.

FIG. 1C represent the wearable skin patch for 3 lead operation with less polymer surface area according to another embodiment.

FIG. 2 represent the Printed Circuit Board (PCB) assembly.

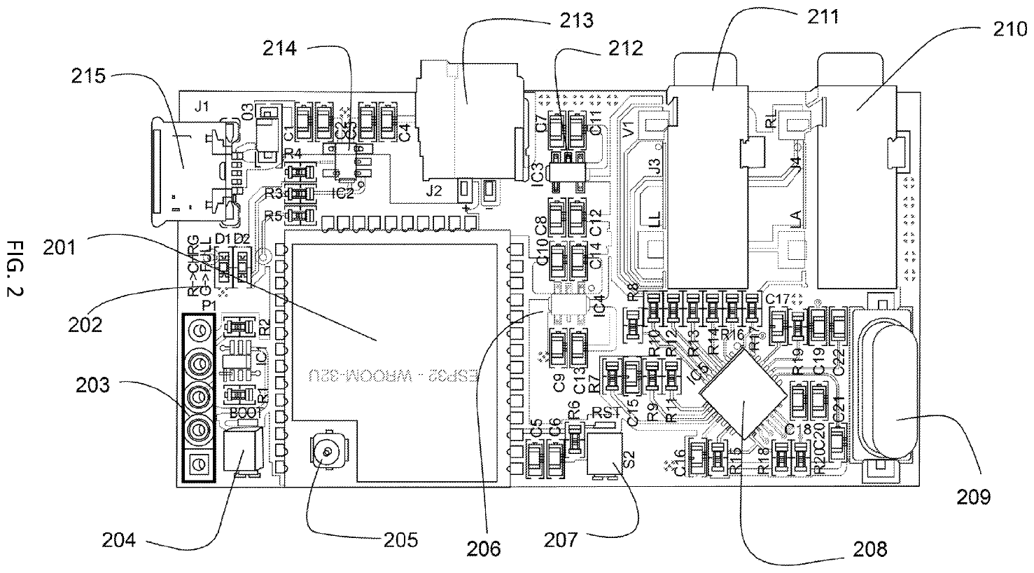

FIG. 3A The plastic casing to hold the PCB and Battery module with female ECG snap connectors according to one embodiment.

FIG. 3B represent the top cover design houses the switches for 3 different modes of operation namely Bluetooth, Wi-Fi and USB according to the embodiment.

FIG. 4 represent the actual positioning of the patch assembly on human body.

FIG. 5 represent the block diagram of the entire hardware to cloud integration system and various communication protocols according to one embodiment.

FIG. 6 represent block diagram of the AI pipeline.

FIG. 7 represent the snapshot of the 3 lead ECG Bluetooth app according to the embodiment.

### DETAILED DESCRIPTION OF THE INVENTION

Detailed embodiments of the present invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representative basis for teaching one skilled in the art to variously employ the present invention in virtually any appropriately detailed structure. Unless defined otherwise, all technical and scientific terms used herein have the same meaning as commonly understood by one of ordinary skill in the art to which the invention belongs.

The present invention overcomes the aforesaid drawbacks of conventional system and method for generation of novel molecules. The objects, features, and advantages of the present invention will now be described in greater detail. Also, the following description includes various specific details and is to be regarded as merely exemplary. Accordingly, those of ordinary skill in the art will recognize that: without departing from the scope and spirit of the present disclosure and its various embodiments there may be any number of changes and modifications described herein.

It must also be noted that as used herein and in the appended claims, the singular forms “a”, “an,” and “the” include plural references unless the context clearly dictates otherwise. Although any systems and methods similar or equivalent to those described herein can be used in the practice or testing of embodiments of the present invention, the preferred, systems are now described.

In this section a detailed description of the various embodiments is provided. The use of the device for accurate prediction of sudden cardiac arrest may also be extended to monitor and diagnose other cardiac diseases. The device may also be used for home based short term to long term ECG recording and also in case of emergency situations related to cardiac arrest. The device may be used for ambulatory monitoring and in-hospital/ICU/Emergency as well.

The main embodiment of the present disclosure is to provide an artificial intelligence enabled wearable ECG skin patch (400) to detect sudden cardiac arrest mainly comprising a wearable ECG skin patch (400) an IoT connected signal transmission unit, and an artificial intelligence engine; wherein,
said wearable ECG skin patch (400) is capable of (and/or arranged to) capture the electrical signal through a flexible printed electronic technology based biocompatible polymer ECG skin patch (400);
said IoT connected signal transmission unit mainly having microcontroller unit (201) being capable of (and/or arranged to) control the signal transmission through the wireless connectivity;
said artificial intelligence engine having an AI and Machine Learning (ML) pipeline that contains a sequence of steps including: a data pre-processing step, a feature extraction step, a feature selection step, a training AI and ML models step, a validation and testing step, and a performance evaluation step;
characterized in that,
said wearable ECG skin patch (400) is capable of (and/or arranged to) capture the entire span of the heart to detect the sudden cardiac arrest; said sudden cardiac arrest being predicted through said AI pipeline, said AI pipeline automate the entire process of accurate cardiac disease prediction from the knowledge database; a peak detector algorithm of said AI engine being capable to capture the instantaneous heart rate from the R-peaks of the ECG and (e.g. to plot the R-R interval time series) to obtain a Heart Rate Variability (HRV).

Another embodiment of the present disclosure is to keep on recording the ECG of a person, in situations where there is no Smartphone or Wi-Fi (312) connection. The device is an integrated expert system that can work in assisting the patient's physician for secondary level of diagnosis and treatment planning. The device is intended to reduce the time and space constraints required in conventional ECG based cardiac monitoring applications. The current available technologies are mostly focused on remote cardiac activity monitoring (615) but providing automated diagnostic information is still a challenge. Most of the devices available in the market fail to provide a one stop solution for preliminary diagnosis of major cardiac issues. The proposed cardiac expert system can really come to the rescue of the people when direct access to medical facilities is a challenge in case of emergency. Since, this device is intended to replace the requirement of a trained expert or cardiologist to be physically present at the point of care, we can speed up the diagnostic process and help the cardiologist to move with his treatment plan as early as possible.

Imagine a situation where a large number of people can stay connected with the cloud management system for quick and accurate diagnosis of cardiac ailments, with this device. The situation where a person's mobility, time and space constraints are all taken into account, and the system is designed to address the needs of the modern times. Although ECG acquisition is a pretty old technology, time and again it has developed from cumbersome arrangement of wires to complete wireless communication with added intelligence. The association of human hearts with AI is something that this device facilitates, as a stunning example of the futuristic cyber-physical world. The various embodiments of the whole system are designed with an intention to present this integrated device, as a significant contribution to the human society which is marching towards a cyber-physical world.

In some embodiments, there is provided a hardware part of the smart device for sudden cardiac arrest prediction as is described in the following section. The various embodiments of the device are referred from the figures and are explained according to their individual functions.

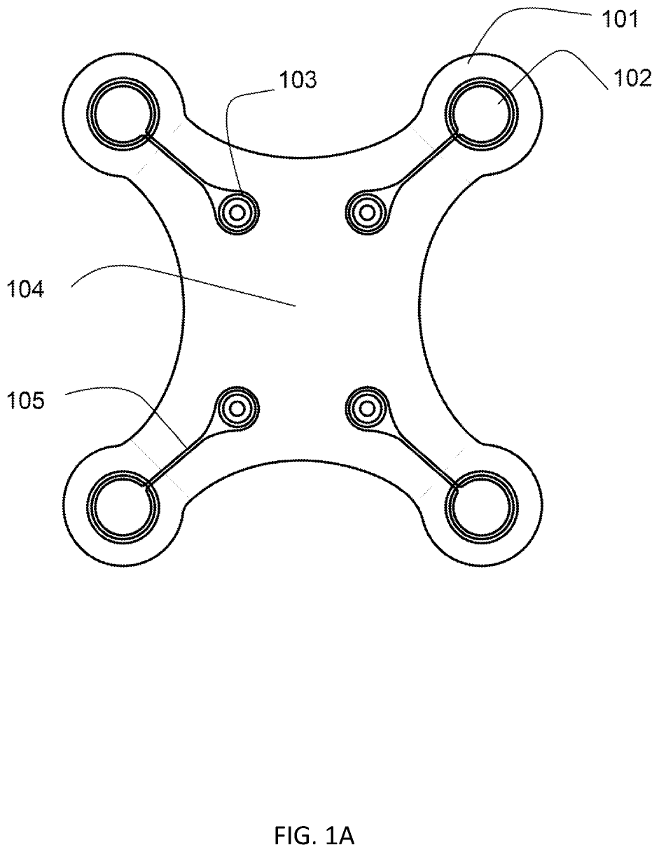

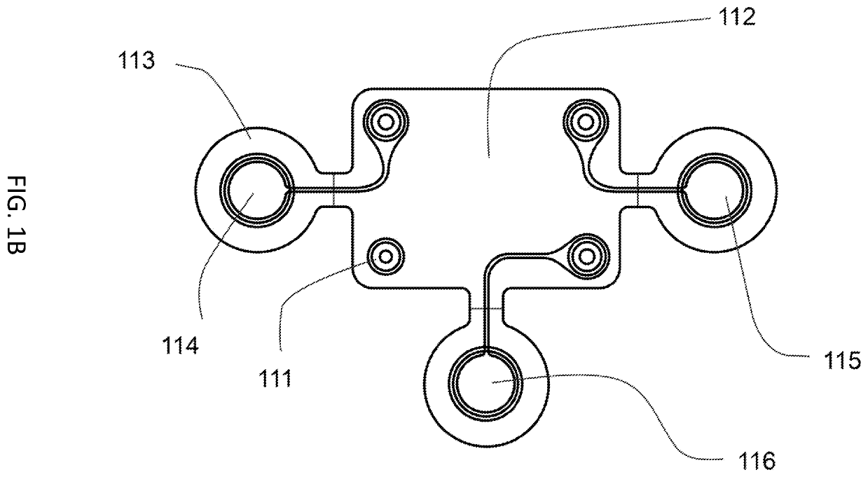

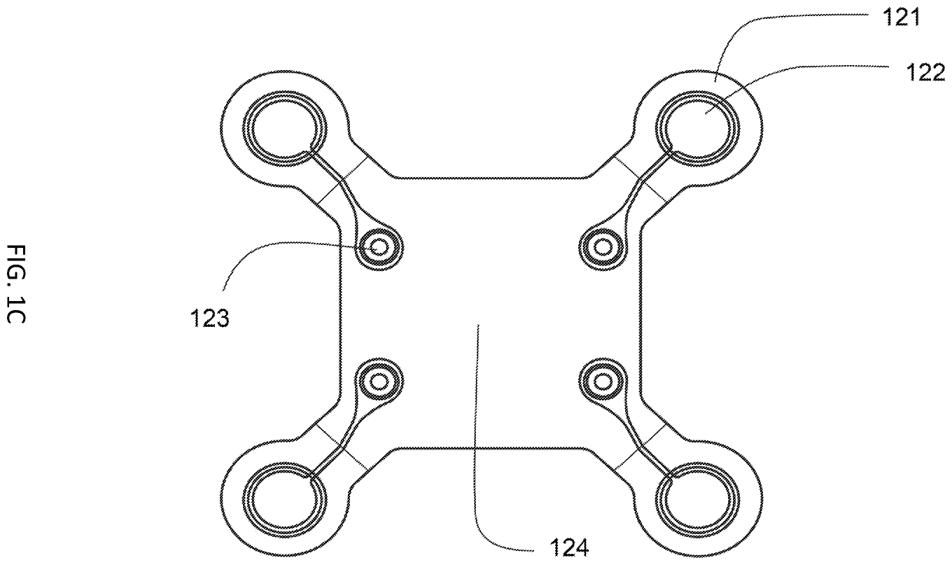

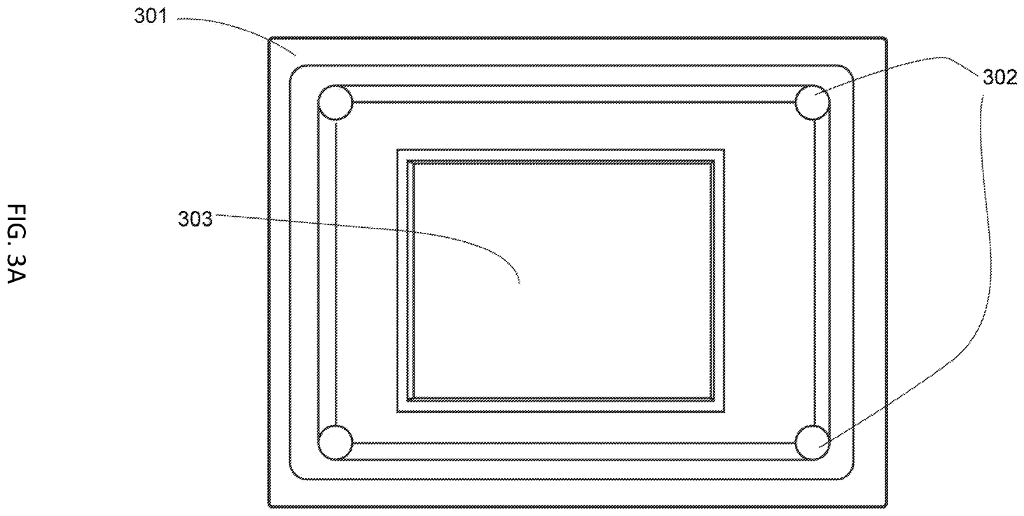

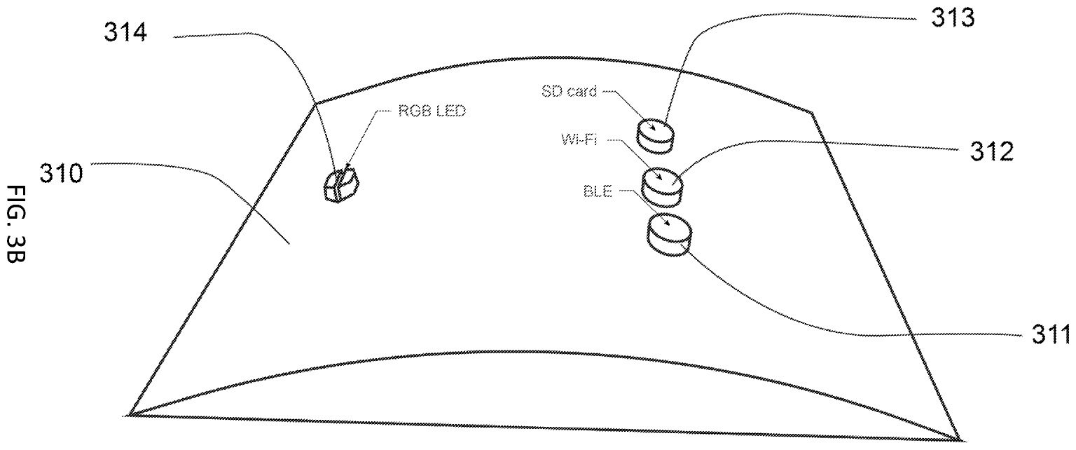

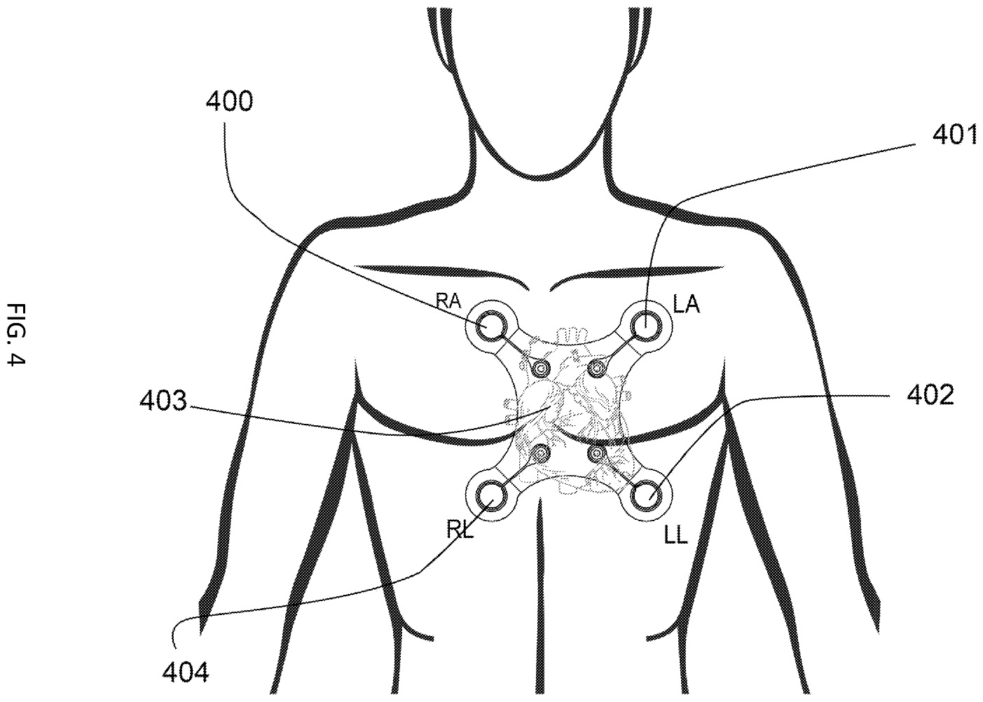

Referring to FIGS. 1A, B and C represent the different 3 lead and single lead operations are shown. The different leads are made to suit patient specific needs according to their ergonomic requirements. In FIG. 1A is the circular area (101, 113,121) that contains the bioadhesive for sticking with the skin. Is the conductive part (102,114, 115, 116, 122) of the patch (400) containing Ag and Ag/Cl based conducting ink which is printed over the TPU substrate (104,112, 124) is copper containing male type snap connectors (103,111,123), that provides the contact point with the PCB assembly box shown in FIG. 3A for signal sensing. The TPU substrate (104,112, 124) laminated with a breathable skin friendly textile material. It gives form and shape to the device. The patch (400) is designed to cover the heart from all four corners as shown in FIG. 4 is the conducting channel (105) of Ag—Agcl ink that is flexible and provides the pathway for the signal to be transmitted to snap connector (103,111,123) points. In another embodiment, FIG. 1B is disconnected from the main circuit assembly but its only function is to hold the PCB assembly box with the skin patch (400).

The single channel or lead ECG data is acquired and embodiments are Right Arm Electrode (114) (RA), Left Arm Electrode (115) (LA) and Right Leg Drive Electrode (116) (RL) respectively. This embodiment may be expanded to work as a three channel device with virtual reference, in future designs. In this arrangement, the PCB assembly box is attached to the patch (400) in a horizontal way. In some embodiments, an attempt to scale down the overall size of the may also be considered. Variations with different inter-electrode (102,114, 115, 116, 122) distance may be employed in future versions. In certain embodiments a different biopolymer may be used to resolve attachment issues, if any.

With reference to FIG. 3, in this embodiment the PCB houses all the active and passive electronic components necessary for signal transduction and transmission. The PCB houses specific circuit combinations for ECG signal sensing, amplification, sampling, storing and transmitting. In embodiment, the Microcontroller unit (MCU) (201) is shown, which is an 80 MHz, dual core processor with inbuilt Bluetooth (311) and Wi-Fi (312) module. The MCU can support high bandwidth data transmission with minimal delay. The MCU is used to drive the entire circuitry is the RED and GREEN LEDs (202) to show the status of battery charge status, where RED indicates charging status and GREEN indicates battery full status is the connector pins (203) for flashing the MCU using USB to UART conversion integrated circuit in module (203), to be connected externally.

In some embodiments, there is provided an embedded firmware that is uploaded then the connector pins module (203) is detached and not required during actual operation is the chip reset switch 204, used for hard resetting the MCU pins and restarting the firmware is the inbuilt Wi-Fi (312) antenna (205) connection point to increase the range of operation in case of weak Wi-Fi (312) zones is the voltage regulator (206, 214) IC to provide 5 V to the AFE circuit (208) in. It draws input 3.7 V from the battery pins connected is the enable switch (207), which drives the pull up register while flashing the firmware on to the MCU. 208 is the low power 3 Channel, 24 Bit, Analog Front End sensing unit for ECG signal is programmed to acquire the signal coming from the input electrode (102,114, 115, 116, 122) arrangement based on the Einthoven triangle and for analog to digital conversion using its inbuilt high resolution 24-bit EA ADC. In a certain embodiment, this AFE (208) may be programmed to activate the right leg drive for better signal quality and reducing grounding errors.

In some embodiments, there is provided an artificial intelligence enabled wearable ECG skin patch (400) to detect a sudden cardiac arrest, the pins through the device being reusable with provision for further firmware updates for up-gradation via offline and online modes. The artificial intelligence enabled wearable ECG skin patch (400) to detect sudden cardiac arrest wherein said ECG skin patch is being capable to automatically plot the data in sync with the circadian rhythm.

In some embodiments, one of the pins may also be accessed for battery voltage level indications. In certain embodiments, the Winston Central terminal may be used as a reference for the precordial ECG leads V1, V2 . . . V6. This AFE (208) is controlled and accessed by the MCU via SPI communication is the crystal oscillator (209) for providing the clock frequency for dataflow synchronization and 3.5 mm female connector (210) audio jacks (210, 211) for connecting the input channels from the ECG female snap connector (302) end. In certain embodiments, these connectors (302) may be replaced with 4 pin connectors (302) with banana clips. In certain embodiments, snap connector (103,111,123) ends may be directly soldered with input tracks of the AFE (208) mounted in the PCB with 10 k resistances. In case of single channel operation only is required is the battery charging circuit (212) IC which also drives the LED (202) indicators of battery charging status. The battery is charged with a micro USB charger using the circuit assembly is the female 2 pin connector (213) for connecting the battery male pins with the PCB is the voltage regulator (206, 214) IC to power regulated 3.7 V to the MCU is the female micro USB connector (215) that charges the battery. In certain embodiments this USB connection (505) may also be configured to drive a USB to UART conversion circuit for flashing the MCU with the firmware.

In FIG. 3A the plastic box for the PCB assembly is shown the laminated outer walls of the box. The outer wall (301) is designed with waterproofing materials, for ultimate protection of the PCB and battery are the 4 ECG female snap connector (103) points for connecting with the male snap connectors (103,111,123) attached to the patch (400) is the battery casing unit (303) to hold the 3.7 V Lithium ion or Coin cells like CR2032. In FIG. 3B the outer cover part (310) of the box assembly is shown. 310 is the outer surface made of plastic and provides protection to the in house components from water or mechanical shock. The top cover is removable to facilitate repair and battery replacement are power cum mode of operation selecting switches, where is for Bluetooth (311), for Wi-Fi (312) and for inbuilt SD card (503, 313) recording.

Referring to FIG. 4, which shows the placement position of the device on the human body is the wearable skin patch (400) with an attached circuit box placed strategically at the points covering the whole heart. The vectors (401, 402, and 403) that represent the voltage difference at Lead I, II and III respectively as per Einthoven triangle is the Right Leg drive point (404) used as the reference to measure the potential differences at Lead I, II and III. The device may be directly applied over the chest as shown after cleaning the contact points of the skin with ethyl alcohol and shaving the hairs for good signal quality. ECG conductive gel may also be applied at the contact points to increase the conductivity between the skin-electrode interfaces. The lower skin impedance is often desired for maximum signal transfer or a matching skin impedance with the input impedance of the op-amp.

In some embodiments, there is provided an application. This part discusses the underlying system architecture of the function of the smart device as an expert system to predict SCA and related heart diseases. An expert system is a set of computer programs designed using AI and a predetermined knowledge base to do human-like interpretation and behaviours.

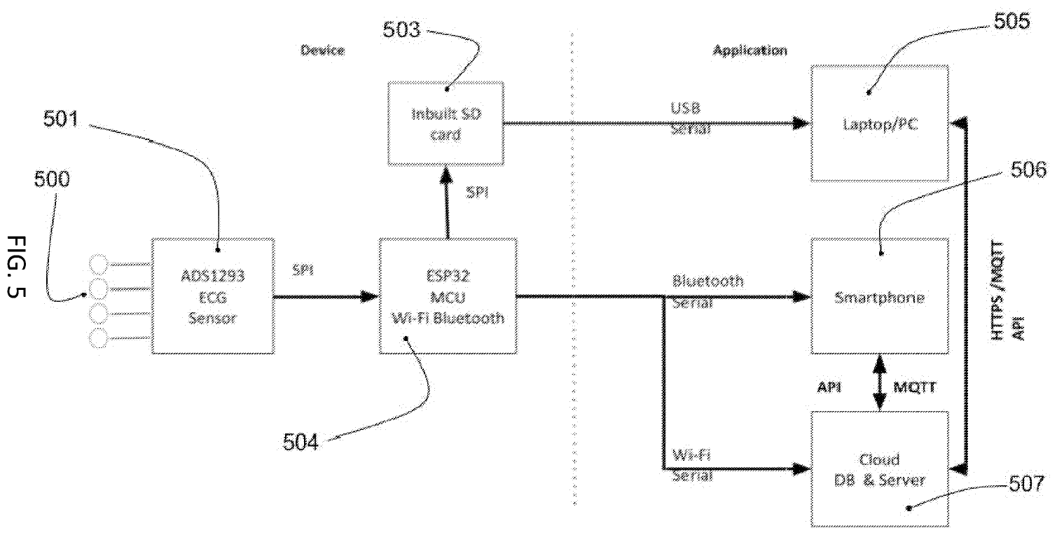

Referring to FIG. 5, the entire IoT system architecture is shown with close emphasis on the hardware to cloud integration and intercommunication between different nodes of the system is the 4 input ECG electrode (500) points entering into AFE with 24 Bit ADC (501) being driven by MCU 504 via SPI communication. When the user selects Bluetooth (311) mode, then the Mobile Application (506) is activated and it starts fetching data from the MCU via Bluetooth (311) communication. The recorded ECG session is being stored in user's mobile memory. The user can upload the saved data into the cloud for offline analysis (507). The backend application in the cloud can fetch the data and compare with available data in cloud DB (503, 507) using a suitable AI model. In an embodiment, when a user selects Wi-FI (312), In that case the MCU (201) will directly connect (504) to the local Wi-Fi (312) gateway and start transmitting data from the device directly to the cloud DB (503). In an Inbuilt SD card (313, 503) module is being shown which is used in one embodiment when the user selects SD card (313, 503) mode. The user can download the data directly into the PC/Laptop using USB connection (505). The data in the cloud can be viewed using a dashboard web application. The APl calls from each node could facilitate the smooth operation of the overall system.

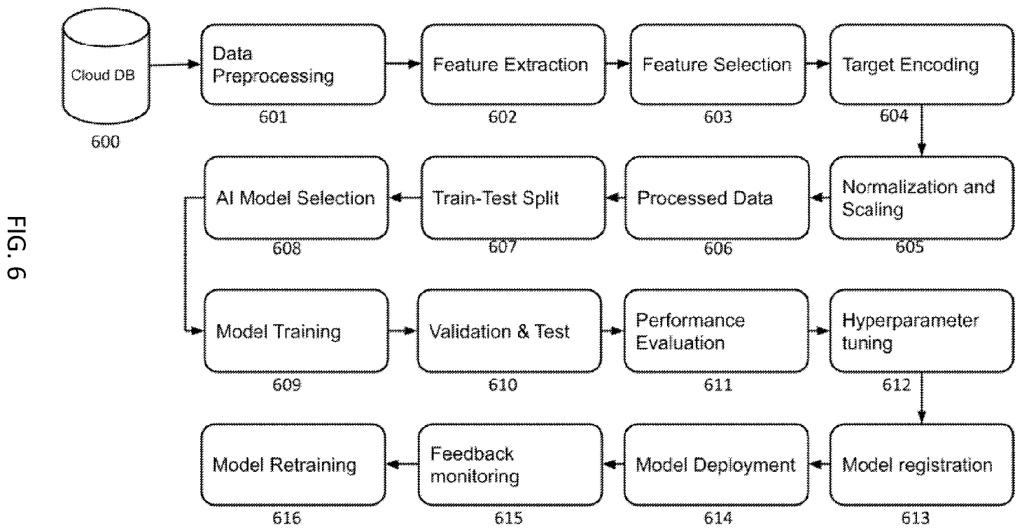

Referring to FIG. 6, where the complete AI pipeline has been shown in connection with the ECG based prediction of SCA and other related cardiac diseases. The pipeline will automate the entire process of accurate cardiac disease prediction from the knowledge database is the entire ECG database (600) with labelled and unlabelled data. In the data pre-processing (601) is to be done, which includes data cleaning, denoising and smoothing. In the ECG features for short term and long term ECG recordings can be extracted (602), which may include HRV, LE, SampEn, FD, QRS segment, Vrms, Peak Counter, MFDFA. From this feature space, in feature selection (603) is being carried out. Feature optimization algorithms like Particle Swarm optimization, Ant Colony Optimization, Genetic Algorithm, Principal Component Analysis. Selecting the optimum features reduces the training time of the AI model. It also helps in improving the training accuracy. In output class labelling or target encoding (604) is done. This is the output layer for any AI model.

Further, the normalisation and scaling of the overall feature space is done so as to convert the values to AI input layer readable formats (605). Now in the present disclosure, to obtain a completely balanced and processed dataset (606). There is performed a test-train split (607) of the dataset in the ratio 80% training set and 20% test set. Then we choose AI models which may include ANN, Support Vector (401, 402, 403) Machines, Random Forest, Gradient descent, Decision Tree and XGboost. In a further stage, we start training the AI model (608) with the training dataset. In this stage the test dataset is uploaded to and fresh data from other sources is selected for validation set. The performance (611) of the AI model is determined using metrics scores such as Accuracy, Precision, Recall, F1 Score, AUC and ROC. A provision is made for tuning the intrinsic parameters of the respective models, to improve the accuracy of the model. This stage of hyper parameter tuning (612) helps in obtaining fast output from the AI models. The model needs to be registered with any cloud service platform with a web server and can make API calls during deployment of the model in stage. The deployed model is monitored in real time and feedback about its actual performance is collected in stage. When the model is deployed in real life applications then there is provision to have access to new and updated dataset. This dataset may be used for model retraining (616) in stage, which will increase the accuracy of the model with time. This entire pipeline would work in a continuous integration/continuous deployment (CI/CD) model.

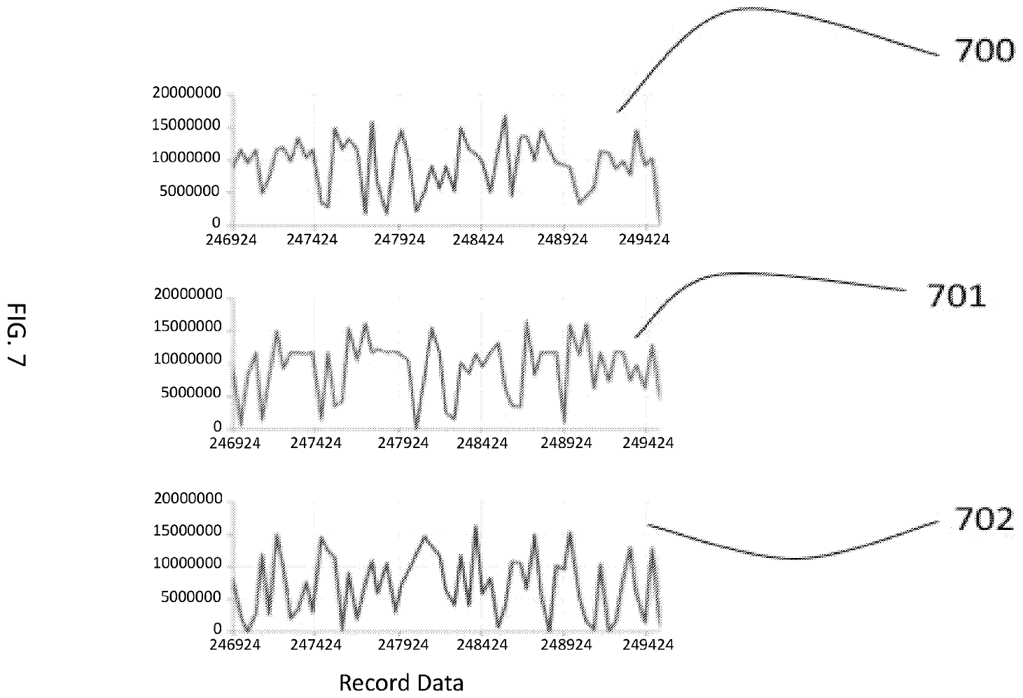

The output of the AI analytics could be accessed on the dashboard or mobile app at user's discretion. The alert message may also be sent to a family physician or caregiver in case of unwanted events. The snapshot of the 3 Channel Acquisition of ECG via Bluetooth (311) using a custom made mobile application (506) is shown in FIG. 7. The FIG. 7 is the recordable data (A, B, C) of the ECG monitoring. Description of the above embodiments summarises the following claims.

Without further description, it is believed that one of ordinary skills in the art can, using the preceding description and the illustrative examples, make and utilize the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

List of Reference Numerals

| | |
|---|---|
| Circular Area (101, 113, 121) | LED (202) |
| Conductive Part (102, 114, 115, | Connecting Pins (203) |
| 116, 122) | Chip Reset switch (204) |
| Snap Connector (103, 111, 123) | Antenna (205) |
| TPU Substrate (104, 112, 124) | Voltage Regulator (206, 214) |
| Conducting Channel (105) | Enable Switch (207) |
| Microcontroller Unit (201) | AFE Circuit (208) |
| Crystal oscillator (209) | Wi-Fi (312) |
| Female connector Audio Jack (210, | SD Card (313, 503) |
| 211) | Wearable Skin Patch (400) |
| Battery charging circuit (212) | Vector (401, 402, 403) |
| Connector (213) | Right leg Drive Point (404) |
| Micro USB connector (215) | Input ECG electrode points (500) |
| Outlets Wall (301) | AFE with 24 Bit ECG Sensor (501) |
| Connector (302) | SPI communication (504) |
| Battery casing unit (303) | USB connection (505) |
| Outer part (310) | Mobile application (506) |
| Bluetooth (311) | Cloud DB Server (507) |

1. An artificial intelligence enabled wearable ECG skin patch (400) to detect sudden cardiac arrest, the skin patch comprising an loT connected signal transmission unit and an artificial intelligence engine; wherein, said wearable ECG skin patch comprises a flexible printed electronic technology based biocompatible polymer ECG skin patch that is capable of capturing an electrical signal;
said loT connected signal transmission unit comprising a microcontroller unit that is capable of controlling signal transmission using a wireless interface;
said artificial intelligence engine comprising an artificial intelligence (Al) and Machine Learning (ML) pipeline that is arranged to perform a sequence of steps comprising: a data pre-processing step, a feature extraction step, a feature selection step, a training step, a validation and testing step, and a performance evaluation step;
wherein,
said wearable ECG skin patch is capable of capturing the entire span of the heart to detect the sudden cardiac arrest; and in that a sudden cardiac arrest is predicted through said Al and ML pipeline;
wherein the Al and ML pipeline is trained using a knowledge database and is thereafter arranged to automate the process of cardiac disease prediction;
wherein a peak detector algorithm of the artificial intelligence engine is capable of capturing the instantaneous heart rate from the R-peaks of the ECG so as to obtain a measure of Heart Rate Variability (HRV), preferably by plotting an R-R interval time series.

2. The artificial intelligence enabled wearable ECG skin patch as claimed in claim 1, further comprising a circular Area containing a bio adhesive which sticking said ECG skin patch with the skin.

3. The artificial intelligence enabled wearable ECG skin patch as claim 1, further comprising a conductive part having flexible dry electrodes which are conducting ink printed over a TPU substrate, preferably wherein said flexible dry electrodes comprise Ag/AgCl ink printed over said Thermoplastic Polyurethane (TPU) substrate.

4. The artificial intelligence enabled wearable ECG skin patch as claimed in claim 3, wherein said TPU Substrate is laminated with textile material in order to provide form and shape to said ECG skin patch.

5. The artificial intelligence enabled wearable ECG skin patch as claimed in claim 1, further comprising one or more of
a snap connector provides the contact point with a PCB assembly box being capable to sensing the signal; and
a conducting channel which provides the pathway for the signal to said ECG patch.

6. The artificial intelligence enabled wearable ECG skin patch as claimed in claim 1, wherein the wireless interface is arranged to communicate using one or more off Bluetooth, WI-FI and/or SD card, and a mobile data network 4G/LTE.

7. The artificial intelligence enabled wearable ECG skin patch as claimed in claim 1, comprising three conducting channels.

8. The artificial intelligence enabled wearable ECG skin patch as claimed in claim 1, comprising, and being powered through, a rechargeable battery.

9. The artificial intelligence enabled wearable ECG skin patch as claimed in claim 1, comprising a plurality of voltage regulators having a diode capable of providing a supply voltage to said integrated circuit.

10. The artificial intelligence enabled wearable ECG skin patch as claimed in claim 1, wherein said loT connected signal transmission unit further comprises a PCB which houses specific circuit combinations for ECG signal sensing, amplification, sampling, storing and transmitting.

11. The artificial intelligence enabled wearable ECG skin patch as claimed in claim 1, wherein said microcontroller unit is capable of driving said PCB components.

12. The artificial intelligence enabled wearable ECG skin patch as claimed in claim 1, wherein said loT connected signal transmission unit further comprises an LED indicator capable of showing the battery level status as well as a critical situation status when abnormal heart activity is sensed.

13. The artificial intelligence enabled wearable ECG skin patch as claimed in claim 1, wherein said loT connected signal transmission unit further comprises one or more of
connector pins being capable of flashing the microcontroller through USB to UART conversion integrated circuit; and
a low power, 3 channel analog front end (AFE) sensing unit for ECG signal.

14. The artificial intelligence enabled wearable ECG skin patch as claimed in claim 13, wherein said AFE is capable of capturing low amplitude multi resolution signals through said wearable skin patch.

15. The artificial intelligence enabled wearable ECG skin patch as claimed in claim 1, wherein said loT connected signal transmission unit further comprises one or more of
a crystal oscillator capable of providing the clock frequency for dataflow synchronisation;
female connector audio jacks for connecting the input channels from the ECG female snap connector end; and
a battery charging circuit IC capable of charging the battery.

16. The artificial intelligence enabled wearable ECG skin patch as claimed in claim 1, wherein said loT connected signal transmission is capable of one or more of storing the data, and processing and analytics, wherein a cloud computing infrastructure is realised with virtual servers and databases with Hypertext Transfer Protocol Secure (https) and Message Queuing Telemetry Transport (mqtt) based communication protocols.

17. The artificial intelligence enabled wearable ECG skin patch as claimed in claim 1, wherein:
said PCB assembly is covered with laminated box; and/or said ECG skin patch comprises four ECG female snap connector points for connecting with the male snap connectors attached to said ECG patch.

18. The artificial intelligence enabled wearable ECG skin patch as claimed in claim 1, wherein said artificial intelligence engine is capable of observing de-noising by discrete wavelet Transforms (DWT).

19. The artificial intelligence enabled wearable ECG skin patch as claimed in claim 1, wherein said device further comprises an loT system architecture capable of providing interconnection between said device and said application, preferably
wherein the data is stored into the memory via Bluetooth mode, Wi-Fi mode, or SD card mode;
said Bluetooth mode being capable to activate the mobile application to store the data;
said Wi-Fi mode being capable to connect said device to local gateway through which said data being transmitted and stored into the cloud servers;
said SD card mode being capable to store said data into the PC/laptop through the USB connection.

20. The artificial intelligence enabled wearable ECG skin patch as claimed in claim 19, wherein said stored data is analysed and compared with available data through said Al engine.

21. The artificial intelligence enabled wearable ECG skin patch as claimed in claim 1, wherein said machine learning pipeline uses one or more of
a nonlinear discrete dynamical system theory (ND-DST) capable of quantifying the underlying cardiovascular dynamics for effective monitoring of the condition of the heart; and
a fractal dimension being capable to indicate the complexity and irregularity of heart beats from the said ECG skin patch profile, preferably wherein said fractal dimension is capable of identifying the intermittent cluster of PQRST arising during the sudden cardiac arrest.

22. The artificial intelligence enabled wearable ECG skin patch as claimed in claim 1, wherein said ECG skin patch can be operated in single channel and/or 3 channel according to patient requirements.

23. The artificial intelligence enabled wearable ECG skin patch as claimed in claim 1, wherein said pins through the device are capable of reuse with provision for further firmware updates for up-gradation via offline and online modes.

24. The artificial intelligence enabled wearable ECG skin patch as claimed in claim 1, wherein said artificial intelligence engine is capable of being trained through Deep Learning models such as ID Convolutional Neural Network (CNN) for real time SCA prediction.

25. The artificial intelligence enabled wearable ECG skin patch as claimed in claim 1, wherein:
said ECG skin patch is an integrated expert system that can work in assisting the patient's physician or cardiologist for secondary level of diagnosis and treatment planning; and/or
said ECG skin patch is capable of automatically plotting the data in sync with the circadian rhythm; and/or
said ECG skin patch is arranged to capture the entire span of the heart in accordance with the principles of Einthoven Triangle.
